(19) United States
(12) Patent Application Publication
(10) Pub. No.: US 2026/0174569 A1
(43) Pub. Date: Jun. 25, 2026

(54) IMPROVED METHOD FOR AIDING THE MANUFACTURE OF A LIMB PROSTHESIS SOCKET FROM A PROVISIONAL PROSTHESIS AND SYSTEM IMPLEMENTING THE METHOD

(71) Applicant: VYTRUVE, Cesson Sevigne (FR)

(72) Inventor: Erwan CALVIER, Cesson Sevigne (FR)

(73) Assignee: VYTRUVE, Cesson Sevigne (FR)

(21) Appl. No.: 19/124,800

(22) PCT Filed: Aug. 3, 2023
(86) PCT No.: PCT/EP2023/071612

§ 371 Date: Apr. 25, 2025

(30) Foreign Application Priority Data
Oct. 26, 2022 (FR) 2211165

(51) Int. Cl. A61F 2/80; A61F 2/50; A61F 2/60; A61F 2/66; A61F 2/72; A61F 2/78

(52) U.S. Cl. CPC A61F 2/80; A61F 2/5046; A61F 2/60; A61F 2/66; A61F 2/72; A61F 2/78; A61F 2/7812; A61F 2002/5049; A61F 2002/5053

(57) ABSTRACT

An improved method for manufacturing a so-called final socket from a provisional socket, for equipping a residual limb of an amputated person. The method has a 3D modelling of the interior surface of the provisional socket that includes a 3D model of said surface in a spatial reference frame and an automatic modification of said 3D model to optimise the arrangement of an artificial limb on the final socket. The method furthermore includes optimising a digitisation step by determining an improved position of a distance-measuring tool in the provisional socket during the modelling. The invention also relates to a system for aiding the manufacture of a final socket configured to implement the method that is the object of the invention.

### TECHNICAL FIELD

The present invention relates to an improved method for manufacturing a limb prosthesis socket. At least one embodiment relates to defining one or more attachment surfaces between a socket printed in accordance with a 3D printing mode and an artificial limb; and at least one embodiment relates to an improved method for obtaining a digital model of a provisional prosthesis socket.

### PRIOR ART

Manufacturing a limb prosthesis comprises producing a provisional socket, modelled according to the form of a residual limb, and then, after a phase of testing on the person for whom the prosthesis is intended, manufacturing a so-called “final” socket from the provisional socket. A socket is a part of a limb prosthesis in which the remaining part of an amputated limb is inserted.

The tests performed with the provisional socket aim to retouch or remodel the interior surface of the provisional socket to reduce to the maximum possible extent surfaces with friction of such a nature as to cause a nuisance or even an injury to the user wearing the prosthesis, and to implement a reference adjustment of the joints of the prosthesis, thus making it possible to obtain the best possible comfort in use and also make it possible to obtain, as far as possible, postures of the wearer of the prosthesis in good correlation with their body shape. For example, in the case of a tibial prosthesis, supplying the tibial prosthesis to a wearer requires making a joint adjustment on two or three axes between an artificial connection element (for example an artificial tibia) and an artificial end part (for example an artificial foot), which then determines a position in space of the interior surface of the provisional socket with regard to the aforementioned reference adjustment, and which in some way resembles a static adjustment of an artificial connection or joint (for example an ankle). In such a context, and in producing a final socket of a tibial prosthesis, for example, it may be necessary to adjust securing points of the artificial “tibia” to obtain a best possible adjustment travel at the ankle joint and to be able to obtain a vertical or almost vertical positioning of the connection element (tibia), thus improving the assumption of mechanical forces on the structure of the prosthesis, during use thereof. It should be noted that the same principle applies for upper limb prostheses (forearm or arm, for example) or yet other types of prosthesis (for example a leg).

When the final socket is manufactured, it is very important when working to preserve the positioning of the interior surface of the socket in a predefined spatial reference frame so as not to lose the reference adjustments previously made during a test phase involving the wearing of the provisional prosthesis by the wearing user.

Practitioners manufacturing prostheses usually work with equipment aiding the manufacture of a final prosthesis having a rigid support structure, which comprises an adjustable provisional socket support lockable in position in three orthogonal movement directions, and making it possible to resume a reference position after a positive mould of the interior surface of the provisional socket has been produced. The use of this type of three-dimensional support structure involves making markings on the structure to “memorise” reference positions during work on manufacturing a final socket. The final socket is then produced by applying materials (in particular fibres and resin) to the positive mould, and the assembly of the final socket and the artificial limb that is intended for it is implemented with positioning references provided by the markings previously made on the rigid support structure. Such a method shows itself to be effective but has a major drawback. In the event of inattention and if there is a faulty adjustment or faulty marking, the initial reference adjustments are lost and all the manufacturing steps subsequent to the adjustment of the provisional socket on the wearer must be recommenced.

The situation can be improved.

### DISCLOSURE OF THE INVENTION

One object of the present invention is to propose a method for manufacturing a so-called “final” or “definitive” socket of a limb prosthesis, the method comprising automated steps aimed at reducing the risk of errors and substantially facilitating the operations, with the aim of reducing the manufacturing time.

For this purpose, a method is proposed for manufacturing a final limb prosthesis socket from a provisional prosthesis, the provisional prosthesis comprising a provisional socket configured to be fitted on a residual limb, and an artificial limb, the artificial limb comprising an artificial end part and a connection element (for example tibial) between the artificial end part and the provisional socket as well as an adjustable first so-called “distal fixing” assembly, lockable in position, between the artificial end part and the connection element, and a second so-called “proximal fixing” assembly between said connection element and the provisional socket, to implement conjointly an adjustment and a locking in position of the artificial end part and of the connection element, the method comprising the steps:
defining a reference adjustment of the distal fixing defining a relative position of the interior surface of the provisional socket with respect to a first reference point of the artificial end part when the artificial end part is positioned in a predetermined reference position, in a first reference space defined according to three directions orthogonal to each other.
positioning and securing said socket, coupled to the connection element, in a second reference space defined with respect to said three directions and with respect to a second reference point representing, in the second reference space, said first reference point of the first reference space so that the position of the socket with respect to the first reference point in said first reference space coincides with the position of the socket with respect to the second reference point in said second reference space,
determining, by a control unit and a distance-measuring device operating in the second reference space, a three-dimensional model representing the interior surface of the provisional socket, and each point of which is defined by coordinates in the second reference space, the three-dimensional model being recorded in the form of a set of information, according to a predetermined format,
manufacturing a second socket referred to as the “final socket” from said three-dimensional model determined.

The method according to the invention can furthermore comprise the additional features, considered alone or in combinations:
The method comprises, between the steps aimed respectively at determining said three-dimensional model and manufacturing said final socket from said three-dimensional model, a modification of the three-dimensional model by inserting bearing surfaces for securing the connection element, arranged to allow securing in a predetermined position of the connection element on the final socket when the reference adjustment is reproduced in a prosthesis comprising the final socket.
The positions of the bearing surfaces are determined and configured on the final socket to allow an excursion of the maximum-amplitude adjustment in two opposite directions of each of the adjustment directions, from the reference adjustment.
The distal fixing comprises two parts inserted one in the other, one of which is secured to the artificial end part and the other is secured to the connection element, and each comprising means for locking in position and, operating in combination with means for locking in position the other one from the two parts of the distal fixing, to conjointly implement an adjustment and a locking in position of the end part and connection element.
The artificial end part is in the form of a foot, a leg, an arm or a hand.
Determining a three-dimensional model representing the interior surface of the provisional socket comprises: positioning a support for holding the distance-measuring device on an axis substantially parallel to a longitudinal axis of the provisional socket and then determining a position of said axis, in said second reference space, on which said holding support is positioned.

Another object of the invention is a system aiding the manufacture of a limb prosthesis socket from a provisional prosthesis, the provisional prosthesis comprising a provisional socket configured to be fitted on a residual limb, and an artificial limb, the artificial limb comprising an artificial end part and a connection element between the artificial end part and the provisional socket as well as a first so-called “distal fixing” assembly, to implement conjointly an adjustment and a locking in position of the artificial end part and of the connection element, the system comprising mechanical and electromechanical means as well as electronic circuits configured to, after a reference adjustment of the distal fixing has been defined, defining a relative position of the interior surface of the provisional socket with respect to a first reference point of the artificial end part when the artificial end part is positioned in a predetermined reference position, in a first reference space defined according to three directions orthogonal to each other:
positioning said socket, coupled to the connection element, in a second reference space defined with respect to said three directions and with respect to a second reference point representing, in the second reference space, said first reference point of the first reference space so that the position of the socket with respect to the first reference point in said first reference space coincides with the position of the socket with respect to the second reference point in said second reference space,
determining, by a control unit and a distance-measuring device operating in the second reference space, a three-dimensional model representing the interior surface of the provisional socket, and each point of which is defined by coordinates in the second reference space, the three-dimensional model being recorded in the form of a set of information, according to a predetermined format,
manufacturing a second socket referred to as the “final socket” from said three-dimensional model determined.

The system according to the invention can furthermore comprise the additional features, considered alone or in combinations:
The system comprises electronic circuits configured to implement a modification of the three-dimensional model by inserting bearing surfaces for securing the connection element, arranged to allow securing in a predetermined position of said connection element on the final socket when said reference adjustment is reproduced in a prosthesis comprising the final socket.
The system comprises circuits configured to determine and configure the positions of the bearing surfaces of said final socket to allow a maximum-amplitude adjustment in the two opposite directions of each of the adjustment directions, from the reference adjustment.
The system comprises mechanical means or modules and electronic circuits configured to position a support for holding the distance-measuring device on an axis substantially parallel to a longitudinal axis of the provisional socket and then determining a position of said axis on which the distance-measuring device is positioned in the second reference space.

Another object of the invention is a computer program product comprising program code instructions for executing the steps of the previously described method when said program is executed by a processor, as well as an information storage medium comprising such a computer program product.

### BRIEF DESCRIPTION OF THE DRAWINGS

The features of the invention mentioned above, as well as others, will emerge more clearly from the reading of the following description of an example embodiment, said description being made in relation to the accompanying drawings, among which:

FIG. 1 illustrates schematically an example of a provisional prosthesis, adapted to a lower limb, and the adjustable elements of which have been adjusted to be adapted to a wearer of the prosthesis;

FIG. 2 illustrates schematically a so-called final prosthesis produced from the provisional prosthesis shown in FIG. 1 and the parameters of which have been optimised;

FIG. 3 illustrates schematically a so-called “distal” securing assembly, usually referred to as a “pyramid”, of a provisional or final prosthesis, according to one embodiment;

FIG. 4 illustrates schematically a three-dimensional system for modelling the interior surface of a provisional prosthesis, according to one embodiment;

FIG. 5 illustrates schematically a three-dimensional modelling of the interior surface of a provisional socket as determined by the system shown in FIG. 4;

FIG. 6 illustrates schematically the three-dimensional modelling of the interior surface of a provisional socket already shown in FIG. 5 after automatic modifications, according to one embodiment;

FIG. 7 illustrates schematically a system for manufacturing by 3D printing a final limb prosthesis socket according to one embodiment;

FIG. 8 illustrates schematically details of the modelling system already shown on FIG. 4;

FIG. 9 illustrates schematically a use of the modelling system already shown on FIG. 8, according to one embodiment;

FIG. 10 shows schematically an internal architecture of the system aiding the manufacture of a limb prosthesis;

FIG. 11 is a diagram illustrating an improved method for aiding the manufacture of a final socket and therefore of a final limb prosthesis from a provisional prosthesis, according to one embodiment of the invention; and

FIG. 12 illustrates schematically a system for three-dimensional modelling of the interior surface of a provisional prosthesis, according to one embodiment.

### DETAILED DISCLOSURE OF EMBODIMENTS

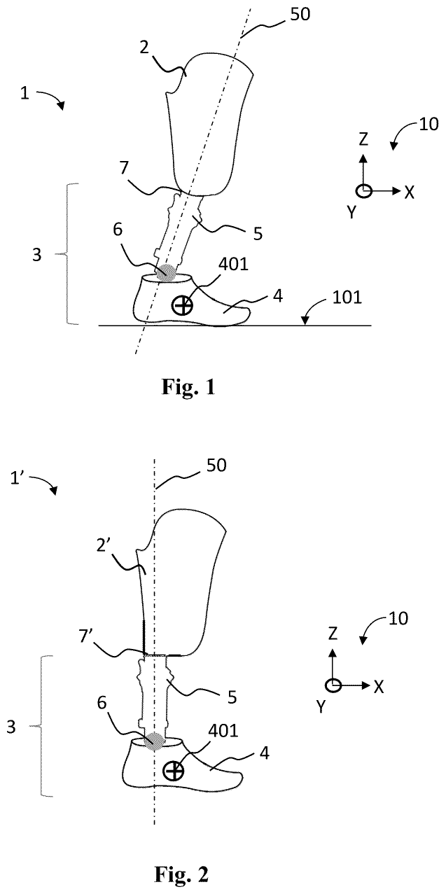

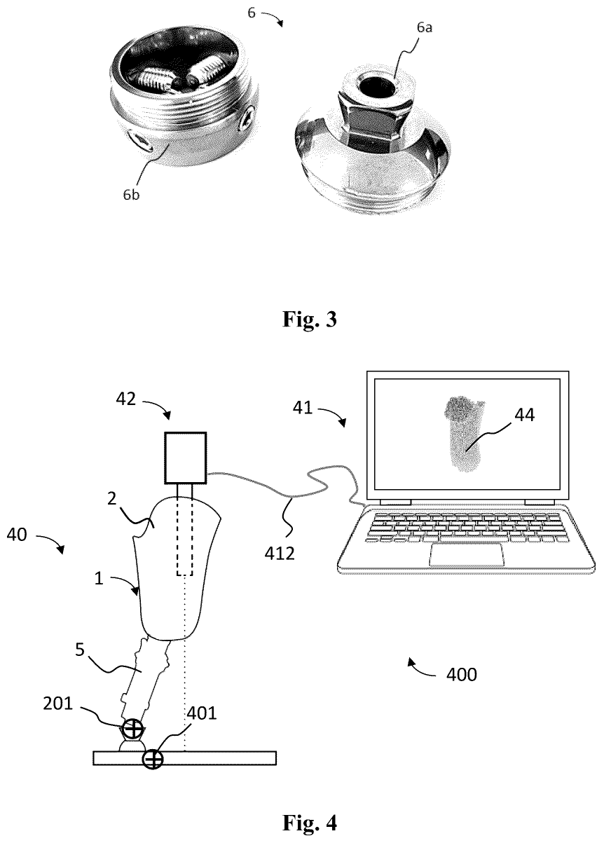

FIG. 1 is a schematic representation of a limb prosthesis 1. According to the non-limitative example shown in FIG. 1, the limb prosthesis 1 is a provisional lower limb prosthesis, also referred to as a tibial provisional prosthesis, comprising an artificial end part 4, also here referred to as “artificial foot”. The provisional prosthesis 1 is said to be provisional in that it comprises a provisional socket 2 fashioned manually by a practitioner to be adapted gradually to the body shape of the wearer of the provisional prosthesis, who must subsequently receive a so-called “final” prosthesis optimised and manufactured from the provisional prosthesis 1. The provisional socket 2 is configured to be fitted on a residual limb of the wearer. The provisional prosthesis 1 comprises an artificial limb 3. The artificial limb 3 comprises the artificial foot 4 and a connection element 5 (an artificial tibia according to the example described here) for mechanically connecting the artificial foot 4 to the provisional socket 2. The artificial limb 3 comprises an adjustable first so-called “distal fixing” assembly 6, of the ball joint type and lockable in position, between the artificial foot 4 and the connection element 5, and a second so-called “proximal fixing” assembly 7, between the connection element 5 and the provisional socket 2. The distal fixing 6 comprises two parts 6a and 6b complementary to each other and uses them inserted one in the other (the parts 6a and 6b of the distal fixing assembly 6 are not detailed in FIG. 1 but are shown in FIG. 3). According to one embodiment, the parts 6a and 6b constitute an assembly usually known by the term “pyramid” used in the field of limb prostheses. The part 6a is secured to the artificial foot 4. The part 6b is for its part secured to the connection element 5. Each of the parts 6a and 6b comprises means for adjusting and locking (blocking) in position, operating in combination with means for adjusting and locking in position the other one from the two parts 6a and 6b of the distal fixing 6. Thus the distal fixing 6 is configured to implement an adjustment and locking in position of the foot 4 and of the connection element 5, by virtue of the conjoint effect of the parts 6a and 6b. FIG. 1 illustrates that, according to the reference adjustments made when the provisional prosthesis 1 is worn by the wearer for whom it is intended, the best adjustment, illustrated in FIG. 1, is such that the securing element 5 has a longitudinal axis 50 inclined in a space referenced by an orthonormal reference frame 10 comprising directions X, Y and Z. The directions X, Y and Z are perpendicular to each other in pairs and such that a plane defined in the directions X and Y is horizontal and the plane defined in the directions X and Z, or in the directions Y and Z, is vertical. The whole of the provisional prosthesis 1 in its position best adapted to the patient, or at the very least considered to be such, is referenced and located in space with respect to a first predefined reference point 401 of the artificial foot 4 when the artificial foot is positioned in a predetermined reference position, for example when the artificial foot 4 is placed on a reference surface 101 parallel to a plane defined according to the directions X and Y (a horizontal plane).

According to a variant embodiment, the adjustable distal fixing is implemented without using a “pyramid” system as previously described, but by means of one or more pivot connections, or a ball joint connection of any type lockable in position.

According to yet another variant embodiment, the adjustable distal fixing is implemented using a connection element made from a material that is deformable when a force above a predetermined threshold is applied thereto; for example a metal bar deformable by means of specific tools dedicated to making such an adjustment.

It should be noted that determining a predetermined reference position, and therefore a reference adjustment for relative position, with respect to one another, of the arrangement of all the elements that make up the prosthesis, depends on the type of prosthesis and in particular on the type of the artificial end part 4 to be connected to the connection element 5. Thus, for example, a reference position along the chest of a wearer can be determined when the artificial end part is an arm; a reference position with respect to the arm can be determined when the artificial end part 4 is a hand, and so on.

It should be noted furthermore that, according to the type of artificial end part 4 assembled on the prosthesis, the connection element 5 may have numerous varied forms so as to provide structural characteristics implementing all or some of the shoulder, hip, knee or ankle functions, for example, but also more generally to implement a strong connection between a socket adapted to a residual portion of the body on the one hand and to an artificial limb on the other hand.

FIG. 2 shows a final prosthesis 1′, the manufacture of which is advantageously optimised by virtue of the improved method according to the invention and by virtue of a manufacturing-aid system implementing this optimised method. The final prosthesis 1′ comprises the same elements as the provisional prosthesis 1 except with regard to the socket and the proximal fixing 7 replaced by a final proximal fixing 7′. In the final prosthesis 1′, the provisional socket 2 is replaced by a final socket 2′. The securing parameters, and the adjustments of the distal 6 and proximal 7′ fixing assemblies are however different from those of the distal 6 and proximal 7 fixings implemented for the provisional prosthesis 1. The purpose of FIG. 2 is to illustrate one of the advantages enjoyed by the use of the so-called final prosthesis 1′ comprising the so-called final socket 2′. This is because, apart from the strength of the materials, the optimised weight and the robustness, for example, the assembly of the elements of the final prosthesis 1′ aims here to obtain a vertical or substantially vertical positioning of the longitudinal axis 50 of the securing element 5, which makes it possible, in the example of a (tibial) prosthesis described here, to advantageously implement an optimised absorption of the mechanical abutment forces present during the use of the final prosthesis 1′ by its wearer. Furthermore, an adjustment of the form of the final socket 2′ at the proximal fixing 7′, with respect to the form of the provisional socket 2, in proximity to the proximal fixing 7, makes it possible not only to obtain an ability to position the securing element 5 vertically or almost vertically but also to obtain an adjustment of the distal fixing 6 having an adjustment travel (or excursion) more equitably distributed in two opposite directions of one and the same adjustment axis. In other words, the adjustment of the distal fixing 6 can be repositioned “at neutral” because of an adjustment of the form of the final socket 2′, at and in proximity to the proximal fixing 7′, with respect to the form of the provisional socket 2. The adjustment excursions of the distal fixing 6, resulting from the reference adjustment made during reiterated tests with the wearer, are then compensated for by an adjustment of the form of the final socket 2′ at the proximal fixing 7′ and by the configuration of the proximal fixing 7′ that result therefrom, considered overall.

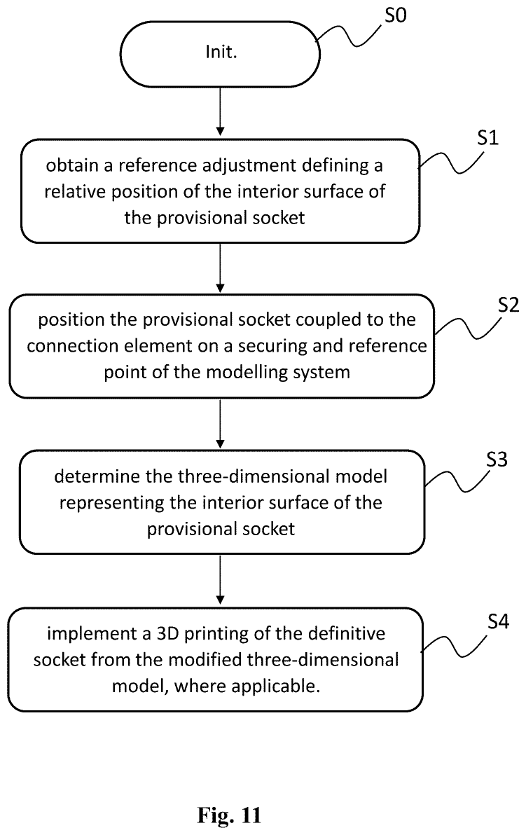

These improvements to the final prosthesis 1′ comprising the final socket 2′, with respect to the provisional prosthesis 1 comprising the provisional socket 2, are cleverly obtained by means of the method according to the invention illustrated in relation to FIG. 11, and which comprises in particular the following successive steps, performed after an initial step S0 of preparing the necessary elements and means:
determining, during a step S1 during which the provisional socket 2 is worn by the wearing user, a reference adjustment of the distal fixing 6 defining a relative position of the interior surface of the provisional socket 2 with respect to a first reference point 401 of the artificial foot 4 when the artificial foot 4 is placed on the reference surface 101, in a first space referenced according to the reference frame 10, also referred to as space 10, defined in the three directions X, Y and Z orthogonal to each other, then
positioning, in a step S2, the provisional socket 2, coupled to the connection element 5, in a second reference space 40 of a system for digitising the interior surface of the provisional socket 2, defined and referenced with respect to three directions X′, Y′ and Z′ of a spatial reference frame 11, respectively parallel to the three directions X, Y and Z of the spatial reference frame 10, and with respect to a second reference point 201 representing, in the second reference space 40 of the digitisation system, the first reference point 401 of the first reference space 10, so that the position of the socket 2 with respect to the first reference point 401 in the first reference space 10 coincides with the position of the socket 2 with respect to the second reference point 201 in the second reference space 40 of the system for digitising the interior surface of the socket 2, and
determining, during a step S3, by a control unit and a distance-measuring device operating in the second reference space 40, a three-dimensional model representing the interior surface of the provisional socket 2, and each point of which is defined by coordinates in the second reference space 40, the three-dimensional model being recorded in the form of a set of information, according to a predetermined format, in the system for digitising the interior surface of the provisional socket 2, and finally
manufacturing, in a step S4, for example by printing in accordance with a 3D printing mode, the second socket 2′, referred to as “final socket”, from the determined (digitised) three-dimensional model, optionally modified.

According to one embodiment, during the manufacturing step S4, the final socket is manufactured by means of a milling method, the milling tool used being controlled digitally by a control unit from a three-dimensional model representing the internal surface of the socket, modified digitally.

According to this example, a milling tool fashions a block of material held in a reference position by means of supports, and implements a gradual removal of material until the final socket 2 determined is obtained.

According to another embodiment, 3D printing and milling operations are combined to manufacture the final socket 2 determined from the three-dimensional model determined and then modified digitally.

In the present description, the terms “digitisation” and “modelling” are indifferently used to describe measurement operations performed on the provisional socket 2 in a reference space and aimed at obtaining information representing a very large number n of points (mesh of points) the respective coordinates Xn, Yn and Zn of which are determined in the reference frame X, Y, Z 10 and recorded to define a digitised three-dimensional model (or 3D model) of the interior surface of the provisional socket 2.

According to one embodiment, the method comprises, cleverly and advantageously, between the steps S2 and S3, a step of calibrated positioning of the distance-measuring device or of an arm of this device carrying a measuring head, so as to be able to insert the distance-measuring head of the distance-measuring device facing any point on the interior surface of the provisional socket 2. To do this, the distance-measuring device is for example cleverly arranged on a ball-joint connection with a three-dimensional structure that carries it, and sensors configured to make rotation measurements on the three rotation axes X′, Y′ and Z′ making it possible to determine the coordinates of distance-measurement points in the second according to the spatial reference frame X′, Y′, Z′, and therefore according to the reference frame X, Y and Z.

The sensors used are for example potentiometers or optical sensors, configured to each make an angular measurement to a tenth of an angular degree. The angular-measurement information obtained by each of the sensors according to three rotation axes then makes it possible to effect a change of 3D reference frame, i.e. to convert coordinates x1, y1, z1 of a point M of the space referenced according to a first orthonormal reference frame X1, Y1, Z1 (or O, i, j, k, for example) into coordinates x2, y2, z2 of the same point M referenced according to a second orthonormal reference frame X2, Y2, Z2 (or P, u, v, w). Cleverly, it is thus possible to modify the position of the tool carrying the distance-measuring sensor, prior to the definition of the three-dimensional model by distance measurements, or even during the making of these measurements, since any point in space can be referenced in a new spatial reference frame defined by modifying the position of the distance-measuring sensor, orientable manually, by means of the angular-measurement sensors combined with the ball-joint connection carrying the distance-measuring sensor support. According to a variant embodiment, the position of the distance-measuring sensor can be controlled digitally (robotic version of the distance-measuring tool) and the changes in spatial reference frame are made according to one and the same coordinate-change principle.

The details of mathematical methods conventionally used for implementing a change in coordinates of a point P referenced in a first orthonormal reference frame (O, i, j, k) into coordinates of the same point P referenced in a second orthonormal reference frame (P, u, v, w) the axes u, v, w of which form respectively angles α, β, γ with the axes i, j, k, and where P is at the coordinates (X, Y, Z) with respect to O, are not developed here since they do not participate in a good understanding of the invention and since a person skilled in the art of mechanical and/or robotic systems is able to make such a change in coordinates for a given point P, and by extension for any point referenced by first coordinates in a first orthonormal reference frame into second coordinates in a second orthonormal reference frame.

FIG. 4 illustrates a positioning of the provisional prosthesis 1 in a system 400 for modelling the interior surface of the provisional socket 2. The system 400 comprises the second reference space 40 defined by the directions X′, Y′ and Z′ respectively parallel to the directions X, Y and Z of the first reference space 10, as well as a support comprising the reference point 201.

Cleverly, the reference point 201 is included in a distal fixing assembly identical to the distal fixing assembly of the provisional prosthesis 1. According to the example embodiment described, the reference point 201 is implemented by intersecting the adjustment axes of a so-called “pyramid” connection assembly such as the distal fixing assembly 6 illustrated in FIG. 3 and composed of the elements 6a and 6b. The use of a pyramid as a reference point 201 of the system 400 for modelling the interior surface of the provisional socket 2 advantageously makes it possible to implement a securing of the connection element 5 to which the provisional socket 2 is secured while keeping the positional adjustment of the connection element 5 with respect to the spatial reference frame 10 (reference adjustment). This is because, if the decoupling of the securing element 5 and of the artificial foot 4 is implemented by unscrewing only two adjacent screws among the four screws of the pyramid system 6, since a new coupling of the connection element 5 is implemented on an element similar to the element 6a of the distal fixing 6, comprising the reference point 201, by re-tightening the two screws previously slackened, the relative positioning of the assembly formed by the connection element 5 and the provisional socket 2 with respect to the spatial reference frame 10 is preserved. This obviously implies that the pyramid element serving as a fixing and comprising the reference point 201 is secured in the securing assembly in a position such that the respective orientation directions of the adjustments in the second reference space 40 are parallel to the directions X, Y and Z of the reference space 10. The modelling system 400 comprises a distance-measuring device 42 connected to a control unit 41 via a bidirectional communication link 412. According to one embodiment, the distance-measuring system 42 is movable and can be moved in the directions X′, Y′ and Z′ of an orthonormal reference frame (spatial reference frame) 11, in the reference space 40. The movements of the distance-measuring device 42 in the reference space 40 can be implemented manually or in an automated manner. That is to say the distance-measuring device 42 can be guided manually by an operator or be guided in the directions X′, Y′ and Z′ by actuators, such as stepper motors, for example under control of the control unit 41 executing software routines provided for this purpose, and comprising a user interface accessible via the control unit 41. In all cases, the modelling system 400 comprises means for determining the precise position in the second reference space 40, by means of a set of position sensors.

Advantageously and according to one embodiment of the invention, the distance-measuring device 42 comprises a rotary arm (or shaft) 421 at the end of which a measuring head 422 is secured (these elements are not shown in FIG. 4 in order to increase the legibility of FIG. 4 but are visible in FIG. 8). According to one embodiment, the measuring head 422 of the measuring device 42 comprises a module for transmitting-receiving a light wave configured to be able to determine a distance between the transmission-reception module and a surface positioned facing the latter, according to a “time-of-flight” determination method. According to one example embodiment, the light wave is a laser ray. Such a configuration advantageously makes it possible to determine a distance between the measuring head and a point on the interior surface of the provisional socket 2, located facing the measuring head 422, when all or part of the arm 421 and the measuring head 422 are inserted in the provisional socket 2. Thus, by means of the modelling system 400, it is possible to determine a model 44 representing the interior surface of the provisional socket 2 in the reference space 40 and therefore consequently in the reference space 10, since the distance between the reference points 401 of the artificial foot 4 of the provisional prosthesis 1 and the securing and reference point 201 is known, and can be expressed in terms of coordinates in the directions X, Y and Z of the reference space 10 or in the directions X′, Y′ et Z′ of the reference space 40. In FIG. 4, the 3D model 44 for representing in space the interior surface of the provisional socket 2 is shown on the screen of the control unit 41, for the purpose of complete illustration of the modelling system 400.

Obviously, the information representing each of the measurement points constituting conjointly modelling points of the interior surface of the socket can be recorded in a working memory of the control unit 41 or in a memory external to the control unit 41 and accessible from the latter.

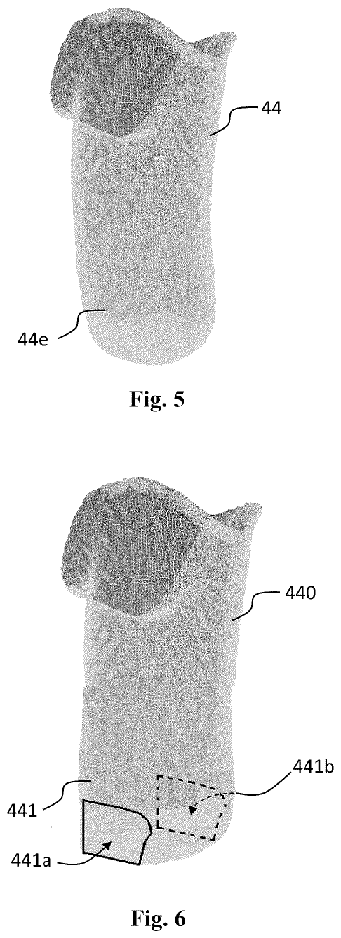

FIG. 5 is an enlarged view of the three-dimensional model 44 representing the interior surface of the provisional socket 2. A lower part 44e represents the surface of the bottom of the provisional socket 2 (or bottom or lower part of the provisional socket 2). Advantageously, it is possible to automatically define by modelling an external surface of a socket to be produced that reproduces the interior surface of the socket 2. According to one embodiment, the control unit 41 executes an algorithm defining a volume corresponding to a thickness around the interior surface 44 modelled and can determine a volume shape to meet specific criteria or given constraints. Thus the control unit 41 defines surfaces for supporting and securing the connection element 5 taking account of the positioning of the interior surface 44 with respect to the reference point 401 of the artificial foot 4. This is made possible by means of the various spatial references used and in particular by means of the use of the securing pyramid for securing the connection element 5 coupled to the provisional socket 2 in the reference space 40, before digitising the interior surface of the provisional socket 2 by means of the detection device 42. FIG. 6 shows a modelling 440 of the final socket 2′ to be produced, obtained from the three-dimensional model 44 of the interior surface of the provisional socket 2. Lateral surfaces 441a and 441b for supporting and securing a lower part 441 of a given volume of the final socket 2′ were determined automatically from the orientation in space, and from the precise position in space, of the interior surface of the socket 2, i.e. in other words according to the position of a limb inserted in the provisional socket 2 with respect to the reference point 201, and therefore finally with respect to the reference point 401 of the artificial foot 4 used during the phase of testing the provisional socket 2, as well as with respect to the reference surface 101 on which the artificial foot 4 rests during at least some of the tests performed. It is thus advantageously possible to reposition the proximal fixing 7′ with respect to the axis 50 of the connection element 5, so as to obtain an assembly of the final socket 2′ and of the connection element 5 that makes it possible to obtain a position of the connection element 5 that is as vertical, or substantially vertical, as possible, during wearing of the final prosthesis 1′ by a wearer, while best satisfying the comfort conditions tested and obtained during the prior phase of testing the provisional prosthesis 1. As a result the final prosthesis 1′ will be as comfortable as possible, while having an optimised configuration of absorbing mechanical forces during use and offering possibilities of well distributed adjustments (excursions substantially equal) in both directions of one and the same axis for adjusting the distal fixing 6.

It is next possible to manufacture the final socket 2′ by means of a 3D printing technique, for example from a three-dimensional model 440 of the final socket 2′, derived from the three-dimensional model 44 of the interior surface of the provisional socket 2, in accordance with the method described. Obviously, the provisional socket can be manufactured by means of another manufacturing technique, from the three-dimensional model 440, for example by milling material by means of a numerical-control milling tool

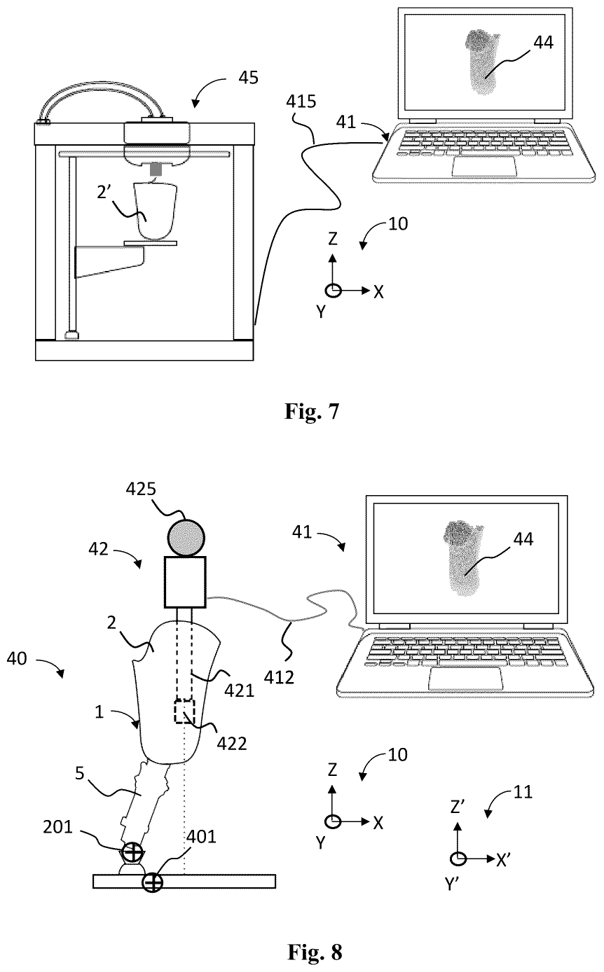

FIG. 7 illustrates a system for manufacturing by 3D printing configured to print the final socket 2′ in three dimensions. The system consists of the control unit 41, used in the modelling system 400, or any similar system, into which the three-dimensional model 440 derived by modifications to the three-dimensional model 44 has been transferred, connected to a 3D printer 45. A bidirectional connection 415 between the control unit 41 and the 3D printer 45 enables the 3D printer 45 to be controlled by the control unit 41 operating under the control of software routines dedicated to this purpose, to print the final socket 2′ in three dimensions from the three-dimensional model 440, and therefore from the three-dimensional model 44 modified by one or more dedicated applications executed by the control unit 41.

FIG. 8 illustrates the system cleverly optimised by means of the manufacture of a final prosthesis 1′ from a provisional prosthesis 1.

According to a preferred embodiment, the distance-measuring device 42 is mounted on a ball-joint connection 425 so as to be able to be directed freely on six axes of freedom. Thus, the distance-measuring device 42 can be moved in rotation and in translation about and along each of the three directions X′, Y′ and Z′. Cleverly, movement sensors make it possible to measure the movements about directions X′, Y′ et Z′, and in translation along these, so as to be able to make measurements in a new spatial reference frame 12, in reference directions X″, Y″ et Z″, or to transpose the results of distance measurements made in the reference space 40 and therefore in the reference space 10, while guaranteeing that the measuring head can access any point on the interior surface of the provisional socket 2. This is particularly advantageous since, in the absence of such a ball-joint connection 425 equipped with movement sensors aimed at measuring in particular rotation movements of the measuring device 42 in each of the three directions X′, Y′ et Z′, certain reference adjustment configurations of the provisional socket 2 do not enable the measuring head 422 to access all the points on the interior surface of the provisional socket 2, or more precisely to be positioned facing any point on this interior surface.

This is in particular the case when the provisional socket 2 is oriented aslant with respect to the vertical in the direction X and/or the direction Y.

FIG. 9 illustrates schematically the advantageous positioning of the distance-measuring device 42 in the provisional socket 2 of the provisional prosthesis 1 by means of the use of a ball-joint connection 425 between the distance-measuring device 42 and the support structure that carries it. By means of the use of rotation sensors incorporated in the ball-joint connection 425 and configured to measure rotations of the distance-measuring device 42 with respect to the spatial reference frame 11, it is possible to make measurements according to a new spatial reference frame 12 (X″, Y″ and Z″) and to convert these measurements into measurements according to the spatial reference frame 11 or according to the spatial reference frame 10.

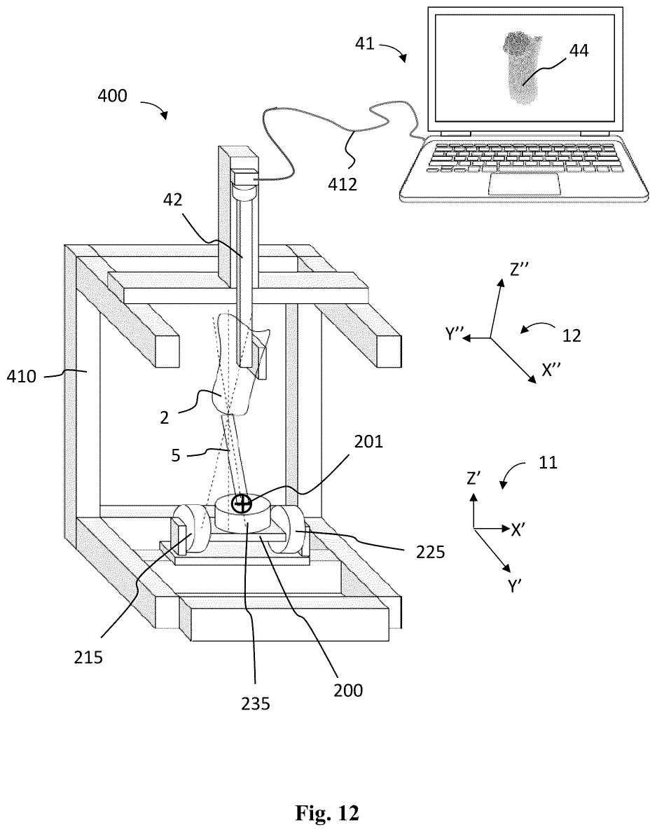

FIG. 12 illustrates a variant embodiment of the system 400 for modelling the interior surface of the provisional socket 2 according to which the distance-measuring device 42 is not mounted articulated on a ball-joint connection (as illustrated in FIG. 8 and FIG. 9, with the ball-joint connection 425), but according to which the reference point 201 is predefined on an articulated support 235 configured to be able to be moved in translation in the two directions X′ and Y′ of the spatial reference frame 11, and in rotation about at least two axes, one of which is oriented parallel to the direction Z′ of the spatial reference frame 11 and the other one of which is oriented parallel to the direction X′ of the spatial reference frame 11. According to this variant, the distance-measuring device 42 is assembled by means of a slider connection equipped with position sensors and can be moved in position, along an axis parallel to the direction Z′ of the spatial reference frame 11. All the elements holding the distance-measuring device 42 and the articulated support 235 are assembled on a frame 410. In this configuration, the arm of the distance-measuring device 42 keeps a fixed position with respect to the direction Z′ of the spatial reference frame 11 and it is the support 235 that can be oriented in rotation about an axis in the direction Z′ and about an axis in the direction X′ by means of mechanical connections of the pivot type each comprising position sensors configured to measure the movement angles of the pivot connections. To do this, the support 235 is assembled on an intermediate support 200, mounted integrally with two lateral pivot connections 215 and 225. According to this variant embodiment also, the distance-measuring device 42 can be guided manually by an operator or be guided in the directions X′, Y′ and Z′ by actuators, such as stepper motors, for example under control of the control unit 41 executing software routines provided for this purpose, and comprising a user interface accessible via the control unit 41. In all cases, there also, the modelling system 400 comprises means for determining the precise position in the second reference space 40 referenced by the spatial reference frame 11, by means of a set of position sensors and the sensors configured to measure the movements of the slider connection carrying the distance-measuring device 42, and the angles of movements made in the pivot connections 215 and/or 225 as well as in the pivot connection of the support 235 make it possible to recalculate the coordinates of all the points of the three-dimensional model 44 in any one of the spatial reference frames 10, 11 or 12, corresponding respectively to the orthonormal reference frames X, Y, Z ; X′, Y′, Z′ and X″, Y″ and Z″. Cleverly, and as in the case of the use of the ball-joint connection 425 previously described in relation to FIG. 8 and FIG. 9, it is thus possible to make measurements according to a new spatial reference frame 12 (X″, Y″ and Z″) and to convert these measurements into measurements according to the spatial reference frame 11 or according to the spatial reference frame 10, which makes it possible to guarantee that the measuring device can make a measurement at any point on the interior surface of the provisional socket 2, which can be used for a 3D modelling whatever the configuration of the provisional prosthesis 1, after the initial reference adjustment.

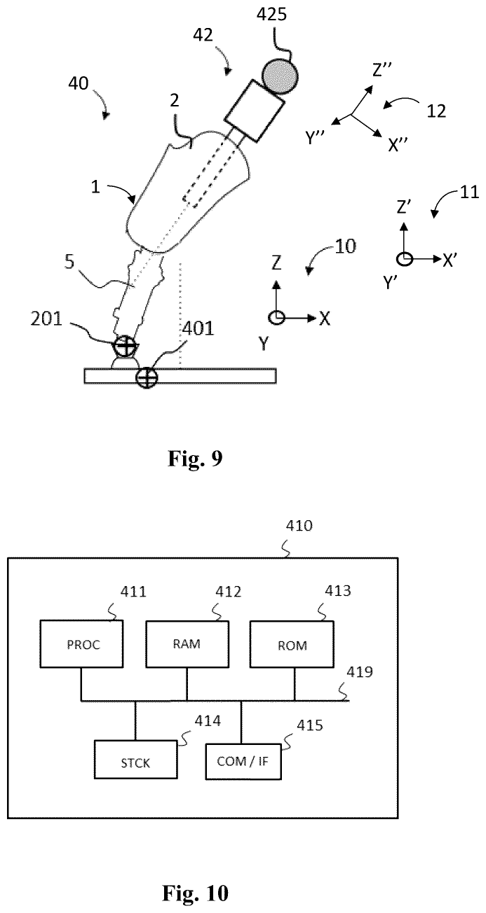

FIG. 10 illustrates schematically an example of internal architecture of the control unit 41. Let us consider by way of illustration that FIG. 10 illustrates an internal arrangement of the control unit 41. It should be noted that the architecture shown could also be used as an internal architecture of the internal systems of the distance-measuring device 42 or as internal architecture of the 3D printing device 45. According to the example of hardware architecture shown in FIG. 10, the control unit 41 then comprises, connected by a communication bus 419: a processor or CPU (“central processing unit”) 411; a random access memory (RAM) 412; a read only memory (ROM) 413; a storage unit such as a hard disk drive (or a storage medium reader, such as an SD (Secure Digital) card reader 414; at least one communication interface 415 enabling the control unit 41 to communicate with other devices to which it is connected, such as the distance-measuring device 42 or the 3D printing device 45 or internal devices such as a screen, a keypad, etc.

According to one embodiment, the communication interface 415 is also configured to control a user interface configured to supervise operations of manufacturing a final socket by means of the system 400 and according to the method described and the variants thereof described.

The processor 411 is capable of executing instructions loaded in the RAM 412 from the ROM 413, from an external memory (not shown), from a storage medium (such as an SD card), or from a communication network. When the control unit 41 is powered up, the processor 411 is capable of reading instructions from the RAM 412 and executing them. These instructions form a computer program causing the implementation, by the processor 411, of all or part of the method described in relation to FIG. 11 or described variants of this method.

All or some of the methods described in relation to FIG. 11 or the described variants thereof can be implemented in software form by executing a set of instructions by a programmable machine, such as a DSP (“digital signal processor”) or a microcontroller, or be implemented in hardware form by a machine or a dedicated component, for example an FPGA (field-programmable gate array) or an ASIC (application-specific integrated circuit). In general, the control unit 41 comprises electronic circuitry configured to implement the methods described in relation to it. Obviously, the control unit 41 furthermore comprises all the elements usually present in a system comprising a digital core implementing functions of a control unit and the peripherals thereof, such as a power supply circuit, a power-supply monitoring circuit, one or more clock circuits, a reset circuit, input/output ports, interrupt inputs and bus drivers, this list being non-exhaustive.

The invention is not limited solely to the embodiments and examples described above and relates more broadly to a method for manufacturing a so-called final prosthesis comprising a so-called final socket from a provisional socket, the prosthesis comprising a connection element between the socket and an end part, a proximal fixing and a settable or adjustable distal fixing.

For example, the socket can be designed and configured to adapt to the shape of a shoulder, an arm, a forearm, a hip, a thigh or a calf and to position itself on the part of the body in question.

Furthermore, the end part connected by means of a connection element present between a proximal fixing and a distal fixing may be a hand, a complete arm provided with a hand, a forearm provided with a hand, a leg provided with a knee, a calf and foot, or a calf provided with a foot, these examples not being limitative.

1. A method for manufacturing a limb prosthesis socket from a provisional prosthesis, the provisional prosthesis comprising a provisional socket configured to be fitted on a residual limb, and an artificial limb, the artificial limb comprising an artificial end part and a connection element between the artificial end part and the provisional socket as well as an adjustable first so-called “distal fixing” assembly, lockable in position, between the artificial end part and the connection element, and a second so-called “proximal fixing” assembly between said connection element and the provisional socket, to implement conjointly an adjustment and a locking in position of the artificial end part and of the connection element, the method comprising the steps:
determining a reference adjustment of the distal fixing defining a relative position of the interior surface of the provisional socket with respect to a first reference point of the artificial part when the artificial part is positioned in a predetermined reference position, in a first reference space defined according to three directions orthogonal to each other.
positioning and securing said socket, coupled to the connection element, in a second reference space defined with respect to said three directions and with respect to a second reference point representing, in the second reference space (40), said first reference point of the first reference space so that the position of the socket with respect to the first reference point in said first reference space coincides with the position of the socket with respect to the second reference point in said second reference space,
determining, by a control unit and a distance-measuring device operating in the second reference space, a three-dimensional model representing the interior surface of the provisional socket, and each point of which is defined by coordinates in the second reference space, the three-dimensional model being recorded in the form of a set of information, according to a predetermined format,
manufacturing a second socket referred to as the “final socket” from said three-dimensional model determined.

2. The method for manufacturing a prosthesis according to claim 1, comprising, between the steps aimed respectively at determining said three-dimensional model and manufacturing said final socket from said three-dimensional model, a modification of said three-dimensional model by inserting bearing surfaces for securing the connection element, arranged to allow securing in a predetermined position of the connection element on the final socket when said reference adjustment is reproduced in a prosthesis comprising said final socket.

3. The method for manufacturing a prosthesis according to claim 2, wherein the positions of said bearing surfaces are determined and configured on said final socket to allow an excursion of the maximum-amplitude adjustment of the distal fixing in each of the adjustment directions, from said reference adjustment.

4. The method for manufacturing a prosthesis according to claim 1, wherein said distal fixing comprises two parts inserted one in the other, one of which is secured to the artificial end part and the other is secured to the connection element, and each comprising means for locking in position and, operating in combination with means for locking in position the other one from the two parts of the distal fixing, to conjointly implement an adjustment and a locking in position of the end part and connection element.

5. The method for manufacturing a prosthesis according to claim 1, wherein said artificial end part is in the form of a foot, a leg, an arm or a hand.

6. The method for manufacturing a prosthesis according to claim 1, wherein determining a three-dimensional model representing the interior surface of the provisional socket comprises:
positioning a support for holding the distance-measuring device on an axis substantially parallel to a longitudinal axis of the provisional socket and determining a position of said axis in said second reference space, so as to be able to implement a change of coordinates of any point on said three-dimensional model from said second reference space into coordinates of a third reference space.

7. A system for aiding the manufacture of a limb-prosthesis socket from a provisional prosthesis, the provisional prosthesis comprising a provisional socket configured to be fitted on a residual limb, and an artificial limb, the artificial limb comprising an artificial end part and a connection element between the artificial end part and the provisional socket as well as an adjustable first so-called “distal fixing” assembly, lockable in position, between the artificial end part and the connection element, and a second so-called “proximal fixing” assembly between said connection element and the provisional socket, to implement conjointly an adjustment and a locking in position of the artificial end part and of the connection element, the system comprising mechanical and electromechanical means as well as electronic circuits configured to:
after a reference setting of the distal fixing has been determined defining a relative position of the interior surface of the provisional socket with respect to a first reference point of the artificial part when the artificial end part is positioned in a predetermined reference position, in a first reference space defined according to three directions orthogonal to each other:
positioning said socket, coupled to the connection element, in a second reference space defined with respect to said three directions and with respect to a second reference point representing, in the second reference space, said first reference point of the first reference space so that the position of the socket with respect to the first reference point in said first reference space coincides with the position of the socket with respect to the second reference point in said second reference space,
determining, by a control unit and a distance-measuring device operating in the second reference space, a three-dimensional model representing the interior surface of the provisional socket, and each point of which is defined by coordinates in the second reference space, the three-dimensional model being recorded in the form of a set of information, according to a predetermined format,
manufacturing a second socket referred to as the “final socket” from said three-dimensional model determined.

8. The system according to claim 7, furthermore comprising electronic circuits configured to implement a modification of said three-dimensional model by inserting bearing surfaces for securing the connection element, arranged to allow securing in a predetermined position of said connection element on the final socket when said reference adjustment is reproduced in a prosthesis comprising said final socket.

9. The system according to claim 8, comprising circuits configured to determine and configure said positions of said bearing surfaces of said final socket to allow a maximum-amplitude adjustment in each of the adjustment directions, from said reference adjustment.

10. The system according to claim 7, furthermore comprising mechanical means, position sensors and electronic circuits configured to:
position a support for holding said distance-measuring device on an axis substantially parallel to a longitudinal axis of said provisional socket and determining a position of said axis on which the distance-measuring device is positioned in the second reference space.

11. (canceled)

12. A non-transitory information storage medium comprising a computer program product that, when executed by a processor, executes the steps of the method according to claim 1.
